(19) United States
(12) Patent Application Publication
(10) Pub. No.: US 2022/0096105 A1
(43) Pub. Date: Mar. 31, 2022

(54) CATHETER BASED DEVICE FOR THE TREATMENT OF CALCIFIED VALVE LEAFLET

(71) Applicant: Pi-Cardia Ltd., Rehovot (IL)

(72) Inventors: Raphael Benary, Tel Aviv (IL); Erez Golan, Rehovot (IL); Oded Meiri, Ram-On (IL); Ronnie Levy, Kochav-Yair (IL)

(73) Assignee: Pi-Cardia Ltd., Rehovot (IL)

(21) Appl. No.: 16/341,924

(22) PCT Filed: Oct. 26, 2017
(86) PCT No.: PCT/IB2017/056657

§ 371 Date: Apr. 15, 2019

Related U.S. Application Data
(60) Provisional application No. 62/412,960, filed on Oct. 26, 2016.

(51) Int. Cl. A61B 17/221; A61F 2/24

(52) U.S. Cl. CPC A61B 17/221; A61B 2017/22098; A61F 2/2418

(57) ABSTRACT

A calcification treatment device includes a catheter with proximal and distal ends. The catheter has at its distal end a non-occluding expansion element movable from a closed to an open position. In the open position, the expansion element is configured to apply force to leaflets of a valve so as to forcefully open the leaflets.

### FIELD OF THE INVENTION

The present invention generally relates to devices and methods for treatment or remodeling of calcified aortic valve leaflets.

### BACKGROUND OF THE INVENTION

Balloon valvuloplasty (BAV) is a well-documented treatment option for patients suffering from aortic stenosis (AS)—a condition which narrows the opening of the aortic valve, restricting blood flow from the left ventricle to the aorta. BAV is a technique whereby an angioplasty catheter having an expandable balloon at its distal end is introduced through the femoral artery and advanced cranially until the balloon element crosses the aortic valve while in its deflated form. The balloon is then inflated using fluid (typically saline) which is forced through the catheter's inflation tube. The balloon enlarges and begins to push the calcified aortic valve leaflets outward in a radial direction, thus theoretically cracking the calcific formations within the leaflets and allowing the valve to regain some of its opening area.

The BAV technique has some critical drawbacks:

Complete occlusion of the aortic valve: As the balloon inflates, the aortic valve is completely blocked, forcing the heart to contract against a complete blockage—a condition which the heart cannot withstand.

Slippage or dislocation of the balloon: This is a two-fold problem that can occur both during positioning of the device and during expansion of the calcified valve leaflets. During systole, the heart undergoes violent motion, which makes positioning of the balloon quite difficult. The balloon has to be situated in such a way that its mid-section is located at the valve annulus so that during inflation, the balloon will not slip from the annulus during inflation. A technique called rapid pacing is used to prevent the movement of the heart to interfere with the BAV procedure and enable complete valve occlusion for short time. This technique involves the electrical stimulation of the heart using pacemaker leads inserted into the heart. The heart rhythm of the patient is then accelerated to over 180 bpm which in fact causes the heart to flutter and thus not to effectually contract. While rapid pacing may help in balloon positioning and toleration of the native valve's occlusion, the added procedure involves added risk, and a small number of patients do not tolerate accelerated pacing very well. In some rare instances, there can be long term myocardial damage due to extended rapid pacing.

Inflation/deflation times: Since the inflation tube of the catheter has a relatively small diameter, the inflation of the balloon is rather lengthy. This fact not only lengthens the procedure unnecessarily, it may also endanger the patient should the physician decide that an immediate deflation of the balloon is required.

Balloon sizing or inflation diameter: A major problem faced by physicians is to fit the correct balloon's inflation diameter. If the selected balloon is oversized or over-inflated, the native valve annulus or leaflets may be torn, resulting in aortic insufficiency—a condition which is particularly dangerous for AS patients.

### SUMMARY OF THE INVENTION

The present invention seeks to provide devices and methods for treating or remodeling of calcified leaflets of a mammalian valve.

The device includes an expandable element placed at the distal end of an intravascular catheter. The expandable device may have a structure made from wire or may be cut from a tube made from a material suitable for this use, including but not limited to, super-elastic material such as nitinol, cobalt-chromium alloy or any type of stainless steel. The structure may also include polymeric material as supporting structure and for controlling the amount of occlusion or opening of the valve area during expansion.

The expansion element is introduced in its crimped or folded state, compressed within said intravascular catheter. The intravascular catheter is introduced into a body lumen, such as the femoral artery, specifically when used to treat a calcified aortic valve. The catheter is advanced cranially through the patient's aorta, until it passes through the aortic valve leaflets. The intravascular catheter may include one or more positioning elements which help locate the device relative to the valve, axially and/or radially, as necessary. The positioning elements may include a plurality of elements which may engage the anatomy surrounding the valve, such as the Valsalva sinuses, thus locking the catheter in the axial direction and preventing the catheter from advancing further into the left ventricle and accurately positioning the expansion element.

The expansion element and the positioning elements are unsheathed when the operator is satisfied with the location of the expansion element relative to the native valve. When unsheathed, the positioning elements deploy to the designed size so as to fit into the Valsalva sinuses, as an example. In another embodiment, the positioning elements may open to an intermediate size, and purposeful action may be required to be taken by the operator to open or expand them to a desired diameter or size. The same may apply for the expansion element.

When the operator is satisfied with the position of the catheter, the expansion element is opened in a generally radial direction, reaching a predetermined diameter. The expansion diameter may be “dialed” in advance using an appropriate feature on the operating handle of the catheter. The operator fully controls the length of time in which the expansion element is open within the native valve complex, since while in the open state, the heart is in valvular insufficiency which may be dangerous to the patient. The expansion element can be immediately reduced in diameter.

The intravascular catheter described herein may have the ability to measure vascular pressure at the distal and proximal end of the expansion element so that an accurate pressure gradient reading on the valve can be obtained during the procedure to assess the treatment effect and adjust the treatment accordingly as necessary. In the case of the treatment of the aortic valve, the intravascular catheter described can measure the pressure within the left ventricle, as well as in the aorta of the patient.

Following the treatment, both the locating assembly and the expansion element are resheathed prior to extraction of the catheter.

Some non-limiting features of some embodiments of the invention include:

a. Expandable mesh device, which does not occlude the aortic annulus when it is in its expanded state

b. Controlled expansion diameter with the ability to adjust as required during the procedure.

c. Radial forces and expansion diameter are controlled through axial movement of the catheter shaft elements.

In some configurations the device includes a positioning assembly to support alignment and precise deployment. The positioning assembly features at least one positioning element.

For example, in the aortic valve the positioning features include three arched arms in a ring-like shape which are adjusted to the sinuses of the aortic valve shape.

In some configurations the device includes a membrane/valve which functions as a temporary valve. While in the expanded mode, the temporary valve function eliminates the regurgitation of blood in the retrograde direction, back into the ventricle.

The steps of deployment are as follows, without limitation:
a. Insert the system, such as via femoral access.
b. Position the expandable mesh for deployment at the site of valve leaflets.
c. Verify the position and alignment, such as by using standard catheter lab visualization tools.
d. Perform mesh expansion against the valve leaflets.

The device of the invention may be used to treat stenosis of a heart valve, instead of balloon valvuloplasty aortic/mitral stenosis procedures. For example, the device may be used in pre-TAVI (transcatheter aortic valve implantation) procedures. The device can improve valve leaflet tissue elasticity and mobility.

While in the expanded mode, the valve leaflets are stretched, thereby increasing leaflet compliance by breaking (e.g., cutting, cracking, grinding, scoring, splitting, etc.) the calcium layer that is on the leaflets.

TAVI implantation quality is highly correlated to the interaction with the contact area of the leaflets tissue. Post expansion, the leaflets may become more compliant, which contributes to the final hemodynamic performance of the implant.

Some advantages of the invention include, without limitation:

The device supports the ability to position and expand supra annular.

The device is non-occlusive so that rapid pacing may not be required.

The device supports controlled and reliable inflation.

The mesh may have a special shape according to required treatment/indication. For example, in the mitral valve the mesh may have a generally elliptical shape.

The device is adjustable to the size of the native annulus. One device can cover a wide range of native valve sizes.

### BRIEF DESCRIPTION OF THE DRAWINGS

The present invention will be understood and appreciated more fully from the following detailed description, taken in conjunction with the drawings in which:

FIG. 1 is a simplified illustration of the valve leaflet treatment device positioned in the aortic valve complex such that the locating elements are located within the Valsalva sinuses and the expansion element is located within the leaflet portion of the aortic valve. The expansion element is in its unopened position.

FIG. 2 is a simplified illustration of the device in its location as described in FIG. 1, with the expansion element in its open position, forcing the valve leaflets to open.

FIG. 3 is a simplified illustration of the valve leaflet catheter located within the aortic valve complex, in a configuration not having a supporting element.

FIG. 4 is a simplified illustration of the distal end of the catheter, specifically its expansion element in its closed position. In this representation, the distal end of the catheter is shown without the positioning element.

FIG. 5 is a simplified illustration of the distal end of the catheter, similar to FIG. 3. The expansion element is in its closed position. Again, in this representation, the distal end of the catheter is shown without the positioning element.

FIG. 6A is a simplified illustration of the expansion element of the valve leaflet treatment catheter in its normally closed yet unsheathed state. An inner polymeric material to control valve opening area during expansion is shown schematically in its closed position within the expansion element. This polymeric material may be formed in a shape of prosthetic valve element as shown in FIGS. 6B and 6C. This configuration does not show the locating element.

FIG. 6B is a simplified illustration of the expansion element of the catheter in its open position. The prosthetic valve element is closed during the diastolic part of the heart cycle. The valve element is closed so as to prevent aortic insufficiency during diastole. The closing of the valve element allows the physician to keep the expansion element in its open position without loading of the left ventricle during diastole.

FIG. 6C is a simplified illustration of the expansion element of the catheter in its open position. The prosthetic valve element is open during the systolic part of the heart cycle. The valve element is open so as to allow forward blood flow during diastole. The opening of the valve element allows the physician to keep the expansion element in its open position without having the left ventricle contract against a complete occlusion, thus eliminating the need for rapid pacing.

### DETAILED DESCRIPTION OF EMBODIMENTS

Reference is now made to FIG. 1, which illustrates a valve leaflet treatment device 30 positioned in the aortic valve complex such that locating (positioning or supporting) elements 34 are located within the Valsalva sinuses and an expansion element 32 is located within the leaflet portion of the aortic valve. The expansion element 32 is in its unopened position. The expansion element 32 is constructed of a mesh or struts with openings formed therein (such as apertures formed in the mesh or gaps between the struts), these openings ensuring that the expansion element 32 is non-occluding, i.e., blood is not impeded from flowing therethrough.

The device is shown after introduction into the vasculature 10 in the aortic arch near the carotid takeoffs 22. The device may include a first (e.g., proximal) shaft 12 and a second (e.g., distal) shaft 14. First shaft 12 may slide over second shaft 14. Both shafts may be delivered over a catheter 16, which in the illustrated embodiment is a pigtail catheter having a proximal portion 18 and a distal portion 20. In one embodiment, catheter 16 passes through the lumen of the second shaft 14 and second shaft 14 passes through the lumen of the first shaft 12. Other arrangements are in the scope of the invention.

The distal portion 20 includes the valve leaflet treatment device 30 positioned against one of the cusps of the aortic valve 24.

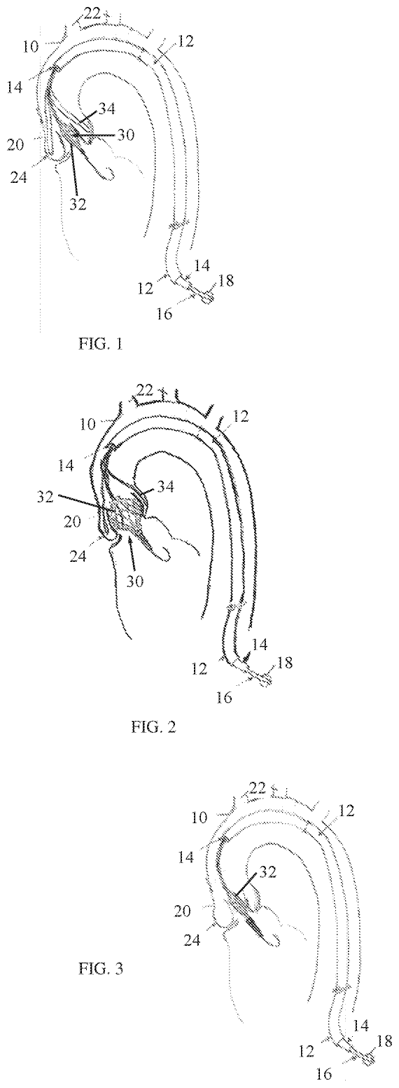

Reference is now made to FIG. 2, which illustrates the device 30 in its location as described in FIG. 1, with the expansion element 32 in its open position, forcing the valve leaflets to open.

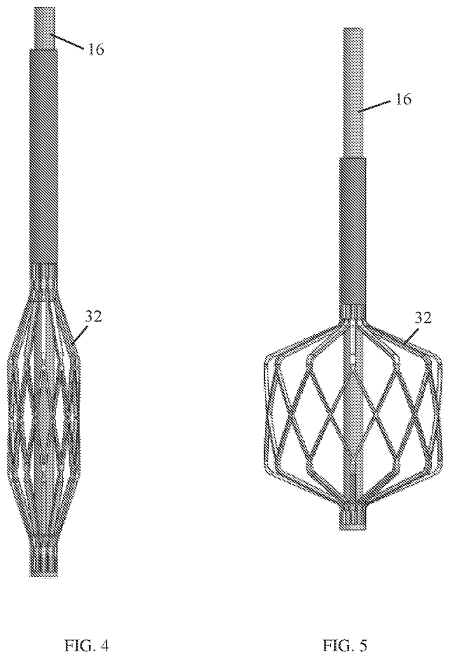

Reference is now made to FIG. 3, which illustrates the valve leaflet catheter 16 located within the aortic valve complex, in a configuration not having a supporting element.

Reference is now made to FIG. 4, which illustrates the distal end of the catheter 16, specifically its expansion element 32 in its closed position. In this representation, the distal end of the catheter 16 is shown without the positioning element.

Reference is now made to FIG. 5, which illustrates the distal end of the catheter 16, similar to FIG. 3. The expansion element 32 is in its closed position. Again, in this representation, the distal end of the catheter 16 is shown without the positioning element.

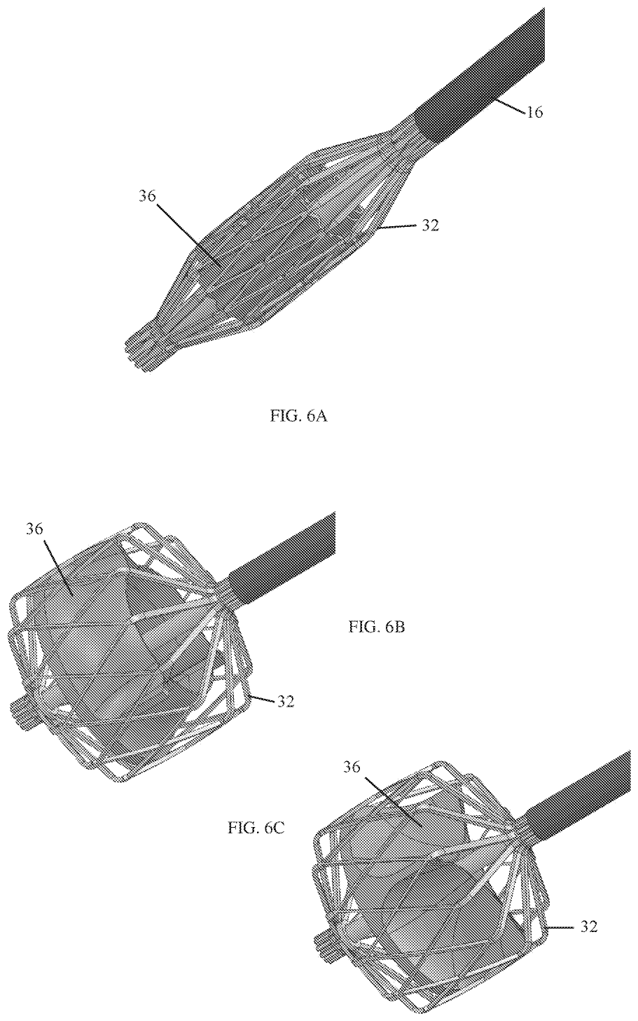

Reference is now made to FIG. 6A, which illustrates the expansion element 32 of the valve leaflet treatment catheter 16 in its normally closed yet unsheathed state. An inner valve element 36 (which may be made of a polymeric material, metallic material or other suitable materials) to control valve opening area during expansion is shown in its closed position within the expansion element 32. Valve element 36 may be formed in a shape of prosthetic valve as shown in FIGS. 6B and 6C. This configuration does not show the locating element.

Reference is now made to FIG. 6B, which illustrates the expansion element 32 of the catheter 16 in its open position. The prosthetic valve element 36 is closed during the diastolic part of the heart cycle. The valve element 36 is closed so as to prevent aortic insufficiency during diastole. The closing of the valve element 36 allows the physician to keep the expansion element 32 in its open position without loading of the left ventricle during diastole.

Reference is now made to FIG. 6C, which illustrates the expansion element of the catheter in its open position. The prosthetic valve element 36 is open during the systolic part of the heart cycle. The valve element 36 is open so as to allow forward blood flow during diastole. The opening of the valve element 36 allows the physician to keep the expansion element 32 in its open position without having the left ventricle contract against a complete occlusion, thus eliminating the need for rapid pacing.

What is claimed is:

1. A calcification treatment device comprising:
a catheter with proximal and distal ends, said catheter having at its distal end a non-occluding expansion element movable from a closed to an open position, wherein in said open position, said expansion element is configured to apply force to leaflets of a valve so as to forcefully open said leaflets, said expansion element being constructed of a mesh or struts with openings formed therein that allow blood and fluids to flow therethrough.

2. The calcification treatment device according to claim 1, wherein said catheter comprises at its distal end a locating element which provides axial and/or radial positioning relative to the valve.

3. The calcification treatment device according to claim 1, wherein the expansion device comprises an internal valve element, located inwards of said mesh or struts, configured to control valve opening during expansion.

4. A method of treating calcification of a heart valve comprising:
placing the device of claim 1 near leaflets of a heart valve; and
expanding said expansion element against the leaflets.

5. The method according to claim 4, comprising using said expansion element to stretch the leaflets, thereby increasing leaflet compliance by breaking a calcium layer that is on the leaflets.

6. The method according to claim 4, wherein said expansion device comprises an internal valve element, located inwards of said mesh or struts, configured to control valve opening during expansion, and the method comprises closing said prosthetic valve element during a diastolic part of a heart cycle and opening said prosthetic valve element during a systolic part of the heart cycle.
